(19) United States
(12) Patent Application Publication
(10) Pub. No.: US 2008/0286387 A1
(43) Pub. Date: Nov. 20, 2008

(54) Standardized method for recovering enriched Indian date extract (IDE), properties of IDE and applications thereof

(76) Inventors: Muhammed Majeed, Piscataway, NJ (US); Rajinder Kumar Bammi, Bangalore (IN); Natarajan Sankaran, Bangalore (IN); Subbafakshmi Prakash, Piscataway, NJ (US); Susmitha Anand, Bangalore (IN); Geetha Kanhangad Gangadharan, Bangalore (IN)

Correspondence Address: SABINSA CORPORATION, 70 ETHEL ROAD WEST, UNIT 6, PISCATAWAY, NJ, 08854, US

(21) Appl. No.: 11/751,033

(22) Filed: May 20, 2007

(51) Int. Cl. A61K 36/889; A61K 131/00; A61P 13/08; A61P 35/00; A61Q 19/00; C07H 1/08

(52) U.S. Cl. 424/727; 536/128

(57) ABSTRACT

Disclosed are (i) a standardized method for the recovery of the polyphenolic contents from the seed coats of Tamarindus indica L. (ii) a standardized polyphenolic extract (10%-90%) from the seed coat of Tamarindus indica L. (standardized Indian date extract) and uses thereof as oral/topical, “skin care” and “hair care” nutra-cosmetic formulations. The standardized Indian Date extract of the present invention exhibits anti-collagenase, anti-elastase, anti-5-alpha-reductase and anti-hyaluronidase activities. The standardized Indian date extract of the present invention is suitable for oral/topical, “skin care” and “hair care” nutra-cosmetic formulations either singly or as “cosmetic blends” with other carefully chosen natural plant actives.

### FIELD OF THE INVENTION

The present invention relates to (i) a standardized method for recovering polyphenolic contents from the seed coats of Tamarindus indica L. (ii) a standardized polyphenolic extract (10%-90%) from the seed coat of Tamarindus indica L. (standardized Indian date extract) and uses thereof as oral/topical, “skin care” and “hair care” nutra-cosmetic formulations.

### BACKGROUND OF THE INVENTION

Tamarindus indica L. or the “tamarind” is the most widely distributed fruit tree of the tropics. Most of its colloquial names are variations on the common English term. In Spanish and Portuguese, it is tamarindo; in French, tamarin, tamarinier, tamarinier des Indes, or tamarindier; in Dutch and German, tamarinde; in Italian, tamarandizio; in Papiamiento of the Lesser Antilles, tamarijn. In the Virgin Islands, it is sometimes called taman; in the Philippines, sampalok or various other dialectal names; in Malaya, asam jawa; in India, it is tamarind or ambli, imli, chinch, puli etc.; in Cambodia, it is ampil or khoua me; in Laos, mak kham; in Thailand, ma-kharm; in Vietnam, me.

Native to tropical Africa, the “tamarind” tree grows wild throughout the Sudan. It was long ago introduced into India that it has often been reported as indigenous there also. It was apparently from India that it reached the Persians and the Arabs who called it “tamar hindi” (Indian date, from the date-like appearance of the dried pulp), giving rise to both its common and generic names. Unfortunately, the specific name, “indica”, also perpetuates the illusion of an Indian origin. The fruit was well known to the ancient Egyptians and to the Greeks in the 4th Century B.C.

The Scientific classification of “tamarind” is as follows.

| Kingdom | Plantae | Plants |
|---|---|---|
| Sub-Kingdom | Tracheobionta | Vascular plants |
| Super Division | Spermatophyta | Seed plants |
| Division | Magnoliophyta | Flowering plants |
| Class | Magnoliopsida | Dicotyledons |
| Subclass | Rosidae | Polypetalous flowers |
| Order | Fabales | Stipulate, compound leaves, |
| | | ten to many stamens which |
| | | are often united by the |
| | | filaments, and a single |
| | | carpel which gives rise to a |
| | | legume (pod) |
| Family | Fabaceae/Leguminoseae | Pea family |
| Genus | Tamarindus L. | Tamarind |
| Species | Tamarindus indica L. | Tamarind |

The present invention focuses on the “tamarind” seeds and extracts thereof that are useful as “skin care” and “hair care” formulations. Prior art references directed towards (i) methods of extracting useful actives from tamarind seeds; and (ii) the cosmetic applications Tamarindus indica are elucidated herein below.

JP7258105 titled “Glucosyl Transferase Inhibitor, Agent for oral cavity and food or drink” to Tamura Kokichi; Fujii Akimi on Oct. 09, 1995 discusses a safe natural substance extracted from tamarind husk with water, monohydric or polyhydric aliphatic alcohol, lower aliphatic ketone, halogenated hydrocarbon, aliphatic hydrocarbon or their mixtures.

JP8231347 titled “Hyaluronidase Inhibitor and Cosmetic” to Kanbara Toshimitsu; Kanechika Rikako; Tamura Kokichi on Sep. 10, 1996 discusses obtaining a hyaluronidase inhibitor containing an extract of tamarind husk as an active ingredient and having high safety.

JP56145955 titled “Production of brown coloring matter by using seed coat of tamarind seed as starting material” to Hasegawa Kanzou on Nov. 13, 1981 discusses a method to obtain a raw material for polyphenol brown coloring matter, by a method wherein the seed coat of tamarind seed is washed with boiling water or a hot aqueous alkaline solution to remove deposited materials and part of a bittering material, and polyphenols are extracted with an alkaline alcoholic solution.

U.S. Pat. No. 6,251,878 titled “Inhibition of UV-induced immune suppression and interleukin-10 production by cytoprotective tamarind oligosaccharides” to Strickland, Faith; Pelley, Ronald; Albersheim, Peter; Darvill, Alan; Pauly, Markus; Eberhard, Stefan on Jun. 26, 2001 discusses cyto-protective tamarind seed xyloglucan oligosaccharides which protect immunological damage of skin exposed to ultra-violet radiations.

The use of tamarind seed extracts, rich in xyloglycans in cosmetic and/or pharmaceutical applications is discussed in U.S. Pat. No. 5,876,729 titled “Use of extracts of tamarind seeds rich in xyloglycans and cosmetic or pharmaceutical product containing such extracts” to Pauly, Gilles on Mar. 02, 1999.

JP11012120 and JP11012155 and titled “Cosmetic”, to Abe Koji; Miyahara Reiji; Nanba Tomiyuki; Uehara Keiichi; Hayashi Toshikatsu; Seki Mochiko on Jan. 19, 1999 discuss a cosmetic excellent in salt resistance, the said cosmetic comprising an alkyl-modified carboxyvinyl polymer with xyloglucan from the seeds of Tamarindus indica L.

JP10167951 titled “Cosmetic” to Ogawa Masazo; Aoki Seiji on Jun. 23, 1998 discusses a cosmetic containing the polysaccharide of Tamarindus indica seeds and a saccharide and/or a polyhydric alcohol, high in safety and excellent in use touch, even when a moisturizing agent is compounded in a large amount.

JP2048517 titled “Hair Tonic” to Komazaki Hisayuki; Miyazawa Kiyoshi; Nakanishi Jiyoutarou; Miyahara Reiji; Uchikawa Keiichi on Feb. 19, 1990 discusses hair tonic, containing one or more extracts of crude drugs, including Tamarindus indica or further one or more surfactants and excellent in promoting effects on prevention of depilation, production, growth of hair etc.

The research publication titled “Isolation and structure elucidation of phenolic antioxidants from Tamarind (Tamarindus indica L.) seeds and pericarp”, authored by Sudjaroen Y, Haubner R, Wurtele G, Hull W E, Erben G, Spiegelhalder B, Changbumrung S, Bartsch H, Owen R W in Food Chem Toxicol. 2005 November; 43(11):1673-82 discusses that tamarind may be an important source of cancer chemo preventive natural products in tropical regions.

The research publication titled “A serine proteinase inhibitor isolated from Tamarindus indica seeds and its effects on the release of human neutrophil elastase”, authored by Fook J M, Macedo L L, Moura G E, Teixeira F M, Oliveira A S, Queiroz A F, Sales M P discusses Proteinaceous inhibitors (PG50) with high inhibitory activities against human neutrophil elastase (HNE) were found in seeds of the Tamarind tree (Tamarindus indica).

In general, the extraction methods discussed herein above include (i) multi-solvent based extraction systems which may be complex; (ii) expensive solvent based extraction systems; (iii) extraction systems that may not very specific or standardized for the polyphenolic contents of the tamarind seed coat; (iv) systems where residual solvents in the extractives may hamper upon the safety profile of the extractives.

Further, the present inventors would like to add upon the existing art on the applications of the Indian Date Extract in “skin care” and hair care” in relation to carefully studied useful properties.

It is thus the principle object of the present invention to develop a standardized extraction system specific for the polyphenolic contents of Tamarindus indica L. which ensures an enriched/concentrated Indian Date extract.

It is another object of the present invention to develop a standardized extraction system specific for the polyphenolic contents of Tamarindus indica L. which is simple and does not involve a multitude of solvent systems.

It is another object of the present invention to develop a standardized extraction system specific for the polyphenolic contents of Tamarindus indica which is not very expensive to operate.

It is another object of the present invention to develop a standardized extraction system specific for the polyphenolic contents of Tamarindus indica which ensures that residual solvents which may affect the safety profile are removed completely from the extractives.

It is another object of the present invention to develop a standardized enriched polyphenolic extract from the seed coats of Tamarindus indica L. (standardized Indian Date Extract)

It is yet another object of the present invention to evaluate the standardized Indian Date extract for its serine protease (elastase and collagenase) inhibitory activity, anti-5-alpha-reductase-activity and anti-hyaluronidase activity.

Further, it is also an object of the present invention to develop oral/topical “skin care” and “hair care” nutra-cosmetic formulations containing the standardized Indian Date extract either singly or as “cosmetic blends” with other carefully chosen natural plant actives.

Still further, the present invention seeks to enhance the scope of “industrial applicability” wherein the cultivation of Tamarindus indica L. for the industrial harnessing of its seed coats will provide numerous employment opportunities to indigenous farmers, harvesters and research personnel.

The present invention fulfills these objectives and provides further related advantages.

### SUMMARY OF THE INVENTION

A standardized method for the recovery of the polyphenolic contents from the seed coats of Tamarindus indica L. is disclosed. A standardized polyphenolic extract (10%-90%) from the seed coat of Tamarindus indica L. (standardized Indian date extract) and uses thereof as oral/topical, “skin care” and “hair care” nutra-cosmetic formulations in relation to useful properties like anti-collagenase, anti-elastase, anti-5-alpha-reductase and anti-hyaluronidase activities are also disclosed. The standardized Indian date extract may be formulated either singly or as “cosmetic blends” with other carefully chosen natural plant actives.

The advantageous features of the present invention include,
a) A standardized extraction system specific for the polyphenolic contents of Tamarindus indica L. ensuring an enriched/concentrated Indian Date extract.
b) A standardized extraction system specific for the polyphenolic contents of Tamarindus indica which is simple in using only methanol as a solvent system and not involve a multitude of solvent systems.
c) A standardized extraction system specific for the polyphenolic contents of Tamarindus indica which is not very expensive to operate.
d) A standardized extraction system specific for the polyphenolic contents of Tamarindus indica which ensures that residual methanol which may affect the safety profile is removed at each step from the extractives.
e) A standardized, enriched polyphenolic extract (10%-90%) from the seed coats of Tamarindus indica L. (standardized Indian Date Extract).
f) A standardized Indian Date extract with excellent inhibitory activity towards serine proteases (elastase and collagenase) and 5-alpha-reductases.
g) Oral/topical “skin care” and “hair care” nutra-cosmetic formulations containing the standardized Indian Date extract either singly or as “cosmetic blends” with other carefully chosen natural plant actives.
h) Oral/topical “skin care” and “hair care” nutra-cosmetic formulations containing the standardized Indian Date extract either singly or as “cosmetic blends” with other carefully chosen natural plant actives which
   (i) Maintain overall skin tone and complexion;
   (ii) Prevent/reduce the pathologies associated with skin aging including but not limited to diminished rate of turn-over of skin cells; reduced skin microcirculation; weak mechanical resistance of the skin due to flattening of the dermal-epidermal junction; formation of superficial or deep/coarse wrinkles, crevices and bumps on the skin; hyper-pigmentation of the skin including the appearance of age spots and freckles; Loss of skin firmness and elasticity leading to sallowness, sagging, blotching, dark under-eye circles and puffy eyes; enlarged skin pores; abnormal desquamation or exfoliation or the skin cells; visible dead skin caused due to flaking and scaling;
   (iii) Exert a sebum control effect;
   (iv) Prevent/reduce acne formation;
   (v) Provide overall hair care; and
   (vi) Prevent/reduce hair fall.
i) Oral nutra-cosmetic formulations containing the standardized Indian Date extract either singly or as “cosmetic blends” with other carefully chosen natural plant actives which additionally offers an alternative therapeutic solution to the problems associated with Benign Prostatic hyperplasia (BPH), prostate cancer, and prostatitis.
j) A standardized extraction method that ensures an enriched/concentrated (10%-90%) polyphenolic extract from the seed coat of Tamarindus indica L. enhancing the scope of “industrial applicability” wherein the cultivation of Tamarindus indica L. for the industrial harnessing of its seed coats will provide numerous employment opportunities to indigenous farmers, harvesters and research personnel.

Other features and advantages of the present invention will become apparent from the following more detailed description, taken in conjunction with the accompanying figures, which illustrate, by way of example, the principle of the invention.

### DETAILED DESCRIPTION OF THE FIGURES

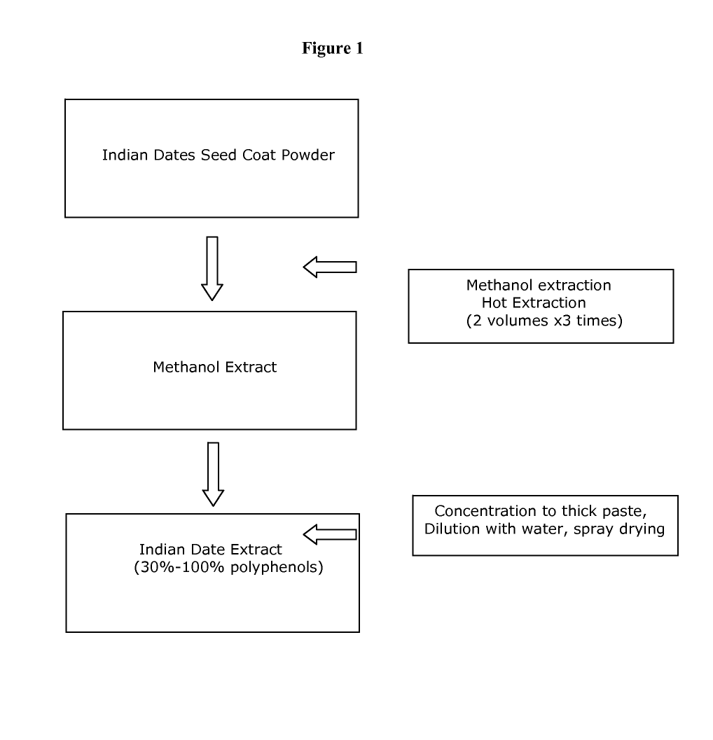

FIG. 1 shows a schematic representation (flow chart) of the steps involved in the standardized extraction method specific for the polyphenolic contents of Tamarindus indica L. ensuring an enriched/concentrated Indian Date extract.

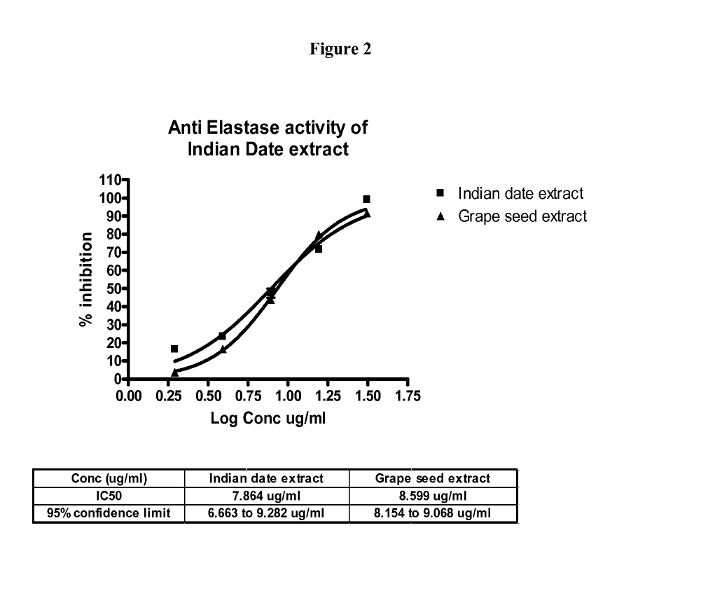

FIG. 2 shows a graphical representation of the comparative percentage inhibition of elastase by the standardized Indian Date Extract and Grape seed extract.

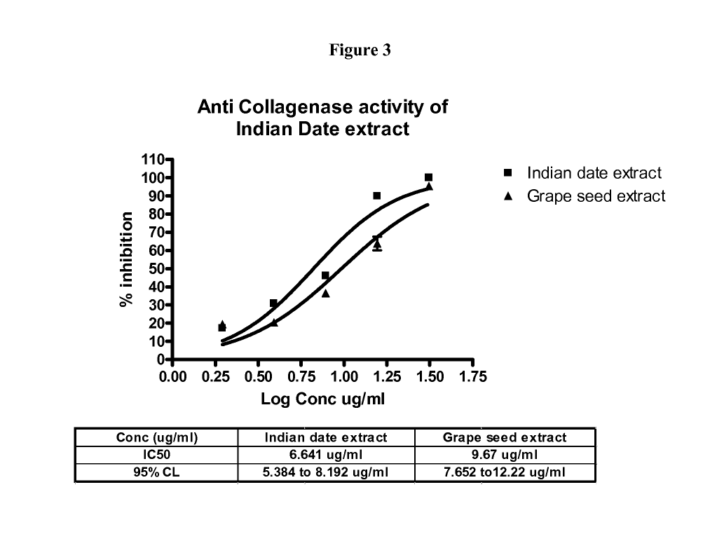

FIG. 3 shows a graphical representation of the comparative percentage inhibition of collagenase by the standardized Indian Date Extract and Grape seed extract

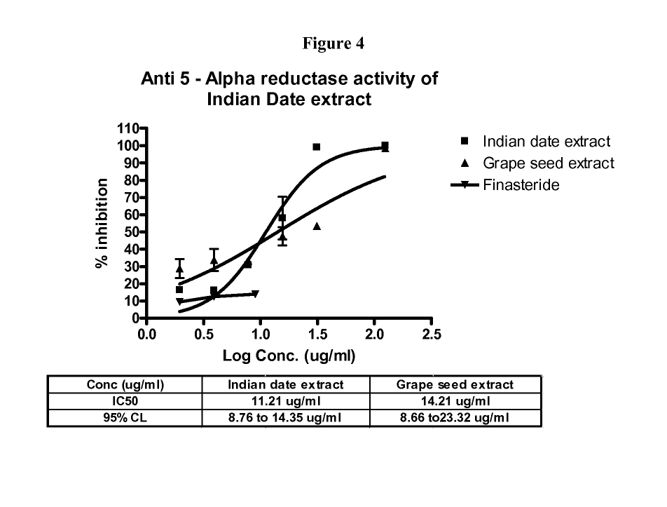

FIG. 4 shows a graphical representation of the comparative percentage inhibition of 5-alpha-reductase by the standardized Indian Date Extract, Grape seed extract and Finisteride

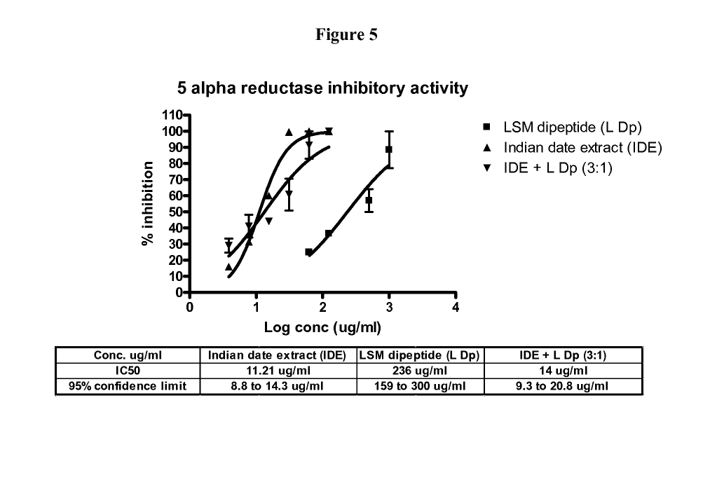

FIG. 5 shows a graphical representation of the comparative percentage inhibition of 5-alpha-reductase by (i) L-selenomethionine (LSM) dipeptide [LDp] alone; (ii) standardized Indian Date Extract (IDE) alone; (iii) IDE:LDp=3:1

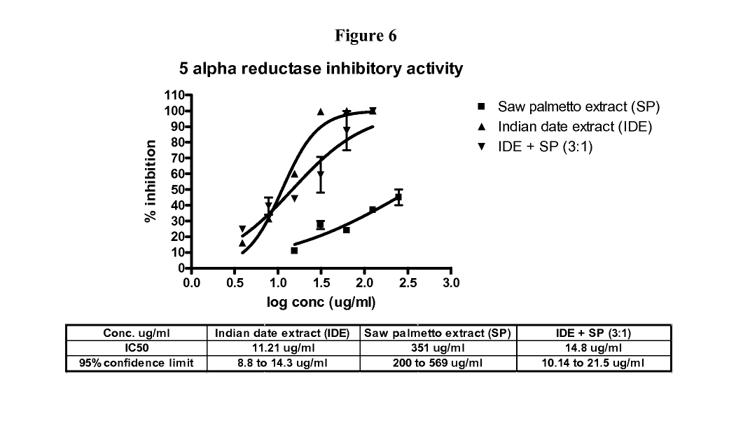

FIG. 6 shows a graphical representation of the comparative percentage inhibition of 5-alpha-reductase by (i) Saw palmetto extract (SPE) alone; (ii) standardized Indian Date Extract (IDE) alone; (iii) IDE: SPE=3:1

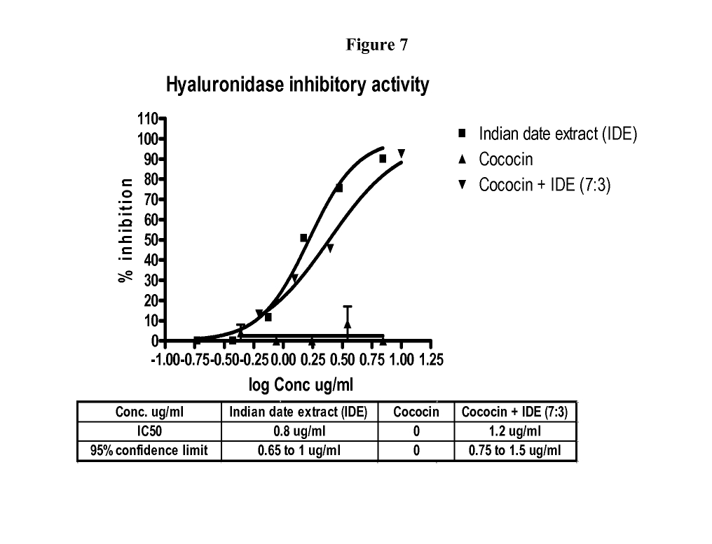

FIG. 7 shows a graphical representation of the comparative percentage inhibition of hyaluronidase by the (i) standardized Indian Date Extract (IDE) alone; (ii) Cococin alone; (iii) IDE: Cococin=7:3

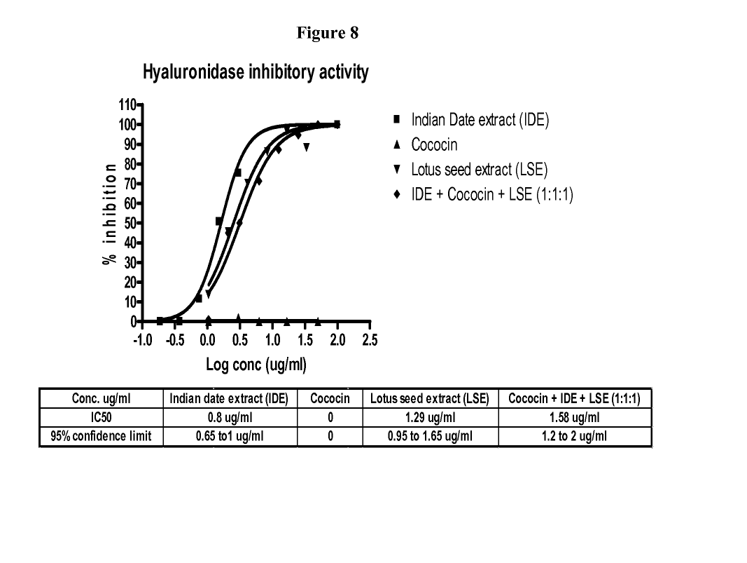

FIG. 8 shows a graphical representation of the comparative percentage inhibition of hyaluronidase by the (i) standardized Indian Date Extract (IDE) alone; (ii) Cococin alone; (iii) IDE: Cococin: Lotus Seed extract (LSE)=1:1:1

### DETAILED DESCRIPTION OF THE MOST PREFERRED EMBODIMENT (FIGS. 1-8)

FIG. 1 shows a schematic (flow chart) representation of the standardized extraction method specific to the polyphenolic contents of the seed coat of Tamarindus indica L. in accordance to the most preferred embodiment of the present invention. The various steps of the said extraction process may be understood from the description presented herein below.
a) Coarsely powdered Tamarind seed coats are charged into a clean reactor.
b) Two volumes of solvent methanol are also charged into the said reactor.
c) The contents in the reactor are refluxed for 3 hours at 60° C. with circulation.
d) The resulting methanol extract from step (c) is drained into a clean container.
e) Steps (b), (c) and (d) are repeated two more times.
f) The methanol extracts resulting from steps (b); (c) and (d) are concentrated into a thick paste under vacuum at 55-60° C.
g) The paste is the dissolved with 1 volume of deionized mineral Water.
h) The residual traces of the solvent, methanol are removed by distilling water under vacuum at 60° C.
i) The aqueous extract resulting in step (h) is then spray dried.

In another most preferred embodiment, an enriched/concentrated (10%-90%) polyphenolic extract (standardized Indian Date Extract) obtained through standardized extraction system elucidated in FIG. 1 is also disclosed.

The experimental protocols and associated results of the study of the useful properties of the standardized Indian Date Extract illustrated in FIGS. 2-8 are set forth as examples herein below.

### EXAMPLE 1

Serine Proteases Inhibition

(a) Elastase Inhibition

Elastase is one of the matrix metalloproteinases, which digests elastin and other components of the extra cellular matrix in the skin. The role of elastase has been implicated in the pathology associated with skin aging. The anti-elastase activity of the standardized Indian Date Extract was measured fluorometrically using the EnzChek Elastase assay. The ability of the standardized Indian Date Extract inhibitor) to inhibit the activity of the elastase enzyme from pig pancreas on the DQ elastin soluble bovine neck ligament (substrate) was fluorometrically assayed in a Microplate reader. The standardized Indian Date Extract showed an IC₅₀ value of 7.864 μg/ml for the elastase enzyme. (FIG. 2)

(b) Collagenase Inhibition

Collagenase is one of the matrix metalloproteinases, which digests collagen and other components of the extra cellular matrix in the skin. The role of collagenase has been implicated in the pathology associated with skin aging. The anti-collagenase activity of the standardized Indian Date Extract was measured fluorometrically using the EnzChek Collagenase assay. The ability of the standardized Indian Date Extract inhibitor) to inhibit the activity of the collagenase Type IV enzyme from Clostridium histolyticum on the DQ elastin from pig skin (substrate) was fluorometrically assayed in a Microplate reader. The standardized Indian Date Extract showed an IC₅₀ value of 6.641 μg/ml for the collagenase enzyme. (FIG. 3)

5-alpha-reductase-activity

FIG. 4 shows a graphical representation of the comparative percentage inhibition of 5-alpha-reductases by the standardized Indian Date Extract and Grape seed extract. The standardized Indian Date Extract showed an IC₅₀ value of 11.21 μg/ml for 5-alpha-reductases. Finasteride which is a 5-alpha-reductase inhibitor showed upto 13% inhibition at a workable concentration of 9 μg/ml.

FIG. 5 shows a graphical representation of the comparative percentage inhibition of 5-alpha-reductases by (i) L-selenomethionine (LSM) dipeptide [LDp] alone; (ii) standardized Indian Date Extract (IDE) alone; (iii) IDE:LDp=3:1. The blend IDE: LDp=3:1 showed an IC₅₀ value of 14 μg/ml.

FIG. 6 shows a graphical representation of the comparative percentage inhibition of 5-alpha-reductases by (i) Saw palmetto extract (SPE) alone; (ii) standardized Indian Date Extract (IDE) alone; (iii) IDE: SPE=3:1. The blend IDE: SPE=3:1 showed an IC₅₀ value of 14.8 μg/ml.

Hyaluronidase Inhibition

In an effort to corroborate the known facts on the anti-hyaluronidase activity of the tamarind seed coat extracts through JP8231343 and JP8231347, and to evaluate the potential of a cosmetic blend to exhibit superior anti-hyaluronidase activity the comparative percentage inhibition of hyaluronidase by the (i) standardized Indian Date Extract (IDE) alone; (ii) Cococin alone; (iii) IDE: Cococin=7:3 was studied as shown in FIG. 7. The standardized Indian Date Extract (IDE) alone showed a promising IC₅₀ value of 0.8 μg/ml for hyaluronidase enzyme while the blend IDE: Cococin=7:3 showed an IC₅₀ value of 1.2 μg/ml. FIG. 8 shows a graphical representation of the comparative percentage inhibition of hyaluronidase by the (i) standardized Indian Date Extract (IDE) alone; (ii) Cococin alone; (iii) IDE: Cococin: LSE=1:1:1. The blend IDE: Cococin: LSE=1:1:1 showed an IC₅₀ value of 1.56 μg/ml.

In additional embodiments, the standardized Indian date extract of the present invention may be formulated either singly or as “cosmetic blends” with other carefully chosen natural plant actives. In preferred embodiments, such formulations may include,
a) Topical/oral “skin care” and “hair care” formulations containing the standardized Indian date extract alone.
b) Topical/oral “skin care” and “hair care” formulations containing the IDE: LDp=3:1 blend.
c) Topical/oral “skin care” and “hair care” formulations containing the IDE: SPE=3:1 blend.
d) Topical/oral “skin care” and “hair care” formulations containing the IDE: Cococin=7:3 blend.
e) Topical/oral “skin care” and “hair care” formulations containing the IDE: Cococin: Lotus Seed extract (LSE)=1:1:1 blend.

In yet another additional embodiment, the standardized Indian date extract of the present invention, suited for oral/topical, “skin care” and “hair care” nutra-cosmetic formulations either singly or as “cosmetic blends” with other natural plant actives as mentioned herein above help to
a) Maintain overall skin tone and complexion
b) Prevent/reduce the pathologies associated with skin aging including but not limited to diminished rate of turn-over of skin cells; reduced skin microcirculation; weak mechanical resistance of the skin due to flattening of the dermal-epidermal junction; formation of superficial or deep/coarse wrinkles, crevices and bumps on the skin; hyper-pigmentation of the skin including the appearance of age spots and freckles; Loss of skin firmness and elasticity leading to sallowness, sagging, blotching, dark under-eye circles and puffy eyes; enlarged skin pores; abnormal desquamation or exfoliation or the skin cells; visible dead skin caused due to flaking and scaling.
c) Exert a sebum control effect
d) Prevent/reduce acne formation
e) Provide an overall “hair care”
f) Prevent/reduce hair fall
g) Exert a therapeutic effect towards the various problems associated with Benign Prostatic hyperplasia (BPH), prostate cancer, and prostatitis.

While the invention has been described with reference to a preferred embodiment, it is to be clearly understood by those skilled in the art that the invention is not limited thereto. Rather, the scope of the invention is to be interpreted only in conjunction with the appended claims.

1. (canceled)

2. A standardized polyphenolic extract (10%-90%) from the seed coat of Tamarindus indica L. (standardized Indian date extract) obtained by an extraction method comprising the steps of:
a) Coarsely powdering Tamarind seed coats;
b) Charging the powdered Tamarind seed coats into a clean reactor;
c) Charging two volumes of solvent (methanol) also into the said reactor;
d) Refluxing the contents of the reactor for 3 hours at 60° C. with circulation;
e) Draining the resulting methanol extract from step (d) into a clean container;
f) Repeating steps (c), (d) and (e) repeated two more times;
g) Concentrating the methanol extracts resulting from steps (c), (d) and (e) into a thick paste under vacuum at 55-60° C.;
h) Dissolving the paste resulting in step (g) with 1 volume of de-ionized mineral Water;
i) Removing the residual traces of the solvent (methanol) by distilling the contents of step (h) under vacuum at 60° C.;
j) and
k) Spray drying the aqueous extract resulting in step (i).

3. The standardized polyphenolic extract (10%-90%) from the seed coat of Tamarindus indica L. (standardized Indian date extract) according to claim 2, wherein the said extract inhibits the activity of serine proteases.

4. (canceled)

5. The standardized polyphenolic extract (10%-90%) from the seed coat of Tamarindus indica L. (standardized Indian date extract) according to claim 2, wherein the said extract inhibits the activity of elastase enzyme with an IC₅₀ value of 7.864 μg/ml.

6. (canceled)

7. The standardized polyphenolic extract (10%-90%) from the seed coat of Tamarindus indica L. (standardized Indian date extract) according to claim 2, wherein the said extract inhibits the activity of collagenase enzyme with an IC₅₀ value of 6.64 μg/ml.

8. (canceled)

9. The standardized polyphenolic extract (10%-90%) from the seed coat of Tamarindus indica L. (standardized Indian date extract) according to claim 2, wherein the said extract inhibits 5-alpha-reductases with an IC₅₀ value of 11.2 μg/ml.

10. (canceled)

11. The standardized polyphenolic extract (10%-90%) from the seed coat of Tamarindus indica L. (standardized Indian date extract) according to claim 2, wherein the said extract inhibits hyaluronidase enzyme with an IC₅₀ value of 0.8 μg/ml.

12. The standardized polyphenolic extract (10%-90%) from the seed coat of Tamarindus indica L. (standardized Indian date extract) according to claim 2, wherein the said extract may be formulated either singly or as “cosmetic blends” with other carefully chosen natural plant actives.

13. A “cosmetic blend” that includes the standardized Indian Date Extract (IDE) of claim 2 and L- Selenomethionine Dipeptide (LDp) in concentration ratios ranging from IDE: LDp=3:1 to 9:1.

14. A “cosmetic blend” that includes the standardized Indian Date Extract (IDE) of claim 2 and Saw Palmetto Extract (SPE) in concentration ratios ranging from IDE: SPE=3:1 to 9:1.

15. A “cosmetic blend” that includes the standardized Indian Date Extract (IDE) of claim 2 and freeze dried coconut water solids COCOCIN in concentration ratios ranging from IDE: COCOCIN=1:9 to 9:1.

16. A “cosmetic blend” that includes the standardized Indian Date Extract (IDE) of claim 2, COCOCIN and Lotus Seed Extract (LSE) in concentration ratios ranging from IDE: COCOCIN : LSE=1:1:1 to 3:2:1.

17. (canceled)

18. The standardized polyphenolic extract (10%-90%) from the seed coat of Tamarindus indica L. (standardized Indian date extract) as in claim 2 in which the said extract prevents/reduces the pathologies associated with skin ageing.

19. (canceled)

20. (canceled)

21. (canceled)

22. The standardized polyphenolic extract (10%-90%) from the seed coat of Tamarindus indica L. (standardized Indian date extract) as in claim 2, in which the said extract exerts anti-acne activity.

23. The standardized polyphenolic extract (10%-90%) from the seed coat of Tamarindus indica L. (standardized Indian date extract) as in claim 2, in which the said extract shows therapeutic potential to counter the problems associated with Benign Prostatic hyperplasia (BPH), prostate cancer, and prostatitis.

24-27. (canceled)
